(19) United States
(12) Patent Application Publication
(10) Pub. No.: US 2004/0077725 A1
(43) Pub. Date: Apr. 22, 2004

(54) Crystalline form of N-(trans-4-isopropylcyclohexane carbonyl)-D-phenylalanine and process for preparation thereof

(75) Inventors: Buchi Reddy Reguri, Hyderabad (IN); Rajasekhar Kadaboina, Hyderabad (IN); Srinivas Polavarapu, Hyderabad (IN)

Correspondence Address: Janet I. Cord, Ladas & Parry, 26 West 61 Street, New York, NY, 10023, US

(73) Assignees: DR. REDDY'S LABORATORIES LIMITED; DR. REDDY'S LABORATORIES, INC.

(21) Appl. No.: 10/649,380

(22) Filed: Aug. 27, 2003

(30) Foreign Application Priority Data
Aug. 28, 2002 (IN) 631/MAS/2002

(51) Int. Cl. A61K 31/198

(52) U.S. Cl. 514/563; 562/450

(57) ABSTRACT

A new crystalline form of nateglinide is provided. The new crystalline form is described by X-ray powder diffraction. Processes for making the new crystalline form of nateglinide are also provided.

### CROSS-REFERENCE TO RELATED APPLICATION

This application claims priority of Indian Patent Application No. 631/MAS/2002, filed Aug. 28, 2002, the disclosure of which is incorporated herein by reference in its entirety.

### BACKGROUND OF THE INVENTION

The drug nateglinide (N-(trans-4-isopropylcyclohexanecarbonyl)-D-phenylalanine) is used in treatment of diabetes. It belongs to the meglitinide class of insulin secretagogues, compounds which stimulate insulin release from the pancreas. Meglitinides tend to be rapid onset compounds with short duration of action, making them particularly suitable for administration just before meals. Preparation of nateglinide and certain of its polymorphic forms is known in the art. However, it is also known that different polymorphic forms of the same drug may have substantial differences in certain pharmaceutically important properties. Therefore, there is a continuing need for new solid forms of nateglinide and new methods of their preparation.

### SUMMARY OF THE INVENTION

In accordance with one aspect, the invention provides a compound which is a crystalline Form X of nateglinide. Preferably, the crystalline Form X of nateglinide has an X-ray diffraction pattern, expressed in terms of 2 theta angles, that includes five or more peaks selected from the group consisting of 3.95±0.09, 4.89±0.09, 5.18±0.09, 6.78±0.09, 7.79±0.09, 10.32±0.09, 13.51±0.09, 14.00±0.09, 16.98±0.09, 17.94±0.09, 18.85±0.09, 19.17±0.09, 20.32±0.09, 21.12±0.09, 22.52±0.09, 23.76±0.09, 24.46±0.09, 27.36±0.09, 28.17±0.09, 30.88±0.09, 31.25±0.09, 32.61±0.09, and 41.65±0.09 degrees. Various embodiments and variants are provided.

In accordance with another aspect, the invention provides a composition that contains nateglinide in a solid form, wherein at least 80% by weight of the solid nateglinide is its crystalline Form X having an X-ray diffraction pattern, expressed in terms of 2 theta angles, that includes five or more peaks selected from the group consisting of 3.95±0.09, 4.89±0.09, 5.18±0.09, 6.78±0.09, 7.79±0.09, 10.32±0.09, 13.51±0.09, 14.00±0.09, 16.98±0.09, 17.94±0.09, 18.85±0.09, 19.17±0.09, 20.32±0.09, 21.12±0.09, 22.52±0.09, 23.76±0.09, 24.46±0.09, 27.36±0.09, 28.17±0.09, 30.88±0.09, 31.25±0.09, 32.61±0.09, and 41.65±0.09 degrees. Various embodiments and variants are provided.

In accordance with yet another aspect, the invention provides a pharmaceutical composition that includes a crystalline Form X of nateglinide and a pharmaceutically acceptable carrier or diluent. Preferably, the pharmaceutical composition is a solid dosage form for oral administration. Various embodiments and variants are provided.

In accordance with yet another aspect, the invention provides a process for making a crystalline Form X of nateglinide, the process including providing a solution of nateglinide in an aromatic hydrocarbon solvent; cooling the solution until a precipitate is formed; and isolating the precipitate, which is the crystalline form X of nateglinide. Various embodiments and variants are provided.

### DESCRIPTION OF DRAWINGS

FIG. 1 shows a sample X-ray powder diffractogram of crystalline Form X of nateglinide.

FIG. 2 is an infrared spectrum of crystalline Form X of nateglinide.

### DESCRIPTION OF PREFERRED EMBODIMENTS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

Unless stated to the contrary, any use of the words such as “including,” “containing,” “comprising,” “having” and the like, means “including without limitation” and shall not be construed to limit any general statement that it follows to the specific or similar items or matters immediately following it. Except where the context indicates to the contrary, all exemplary values are intended to be fictitious, unrelated to actual entities and are used for purposes of illustration only. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

For purposes of the present invention, the following terms are defined below.

The crystalline compound designated herein as “crystalline Form X”, and referred to hereinafter as a crystalline Form X of nateglinide, is a new crystalline polymorph of nateglinide different from known polymorphs. It is characterized via X-ray powder diffraction, DSC and/or infrared spectroscopy.

“Pharmaceutically acceptable” means that which is useful in preparing a pharmaceutical composition that is generally non-toxic and is not biologically undesirable and includes, but is not limited to, that which is customarily utilized for veterinary use and/or human pharmaceutical use.

The term “composition” includes, but is not limited to, a powder, a solution, a suspension, a gel, an ointment, an emulsion and/or mixtures thereof. The term composition is intended to encompass a product containing the specified ingredient(s) in the specified amount(s), as well as any product, which results, directly or indirectly, from combination of the specified ingredients in the specified amounts. A “composition” may contain a single compound or a mixture of compounds. A “compound” is a chemical substance that includes molecules of the same chemical structure.

The term “pharmaceutical composition” is intended to encompass a product comprising the active ingredient(s), pharmaceutically acceptable excipients that make up the carrier, as well as any product which results, directly or indirectly, from combination, complexation or aggregation of any two or more of the ingredients, or from dissociation of one or more of the ingredients, or from other types of reactions or interactions of one or more of the ingredients. Accordingly, the pharmaceutical compositions of the present invention encompass any composition made by admixing the crystalline Form X of nateglinide, additional active ingredient(s), and pharmaceutically acceptable excipients.

The term “excipient” means a component of a pharmaceutical product that is not the active ingredient, such as filler, diluent, carrier, and so on. The excipients that are useful in preparing a pharmaceutical composition are preferably generally safe, non-toxic and neither biologically nor otherwise undesirable, and are acceptable for veterinary use as well as human pharmaceutical use. “A pharmaceutically acceptable excipient” as used in the specification and claims includes both one and more than one such excipient.

“Therapeutically effective amount” means the amount of a compound that, when administered for treating or preventing a disease, is sufficient to effect such treatment or prevention for the disease. The “therapeutically effective amount” will vary depending on the compound, the disease and its severity and the age, weight, etc., of the patient to be treated.

When referring to a chemical reaction, the terms “treating”, “contacting” and “reacting” are used interchangeably herein and refer to adding or mixing two or more reagents under appropriate conditions to produce the indicated and/or the desired product. It should be appreciated that the reaction which produces the indicated and/or the desired product may not necessarily result directly from the combination of two reagents which were initially added, i.e., there may be one or more intermediates which are produced in the mixture which ultimately leads to the formation of the indicated and/or the desired product.

The term “substantially free of” in reference to a composition, as used herein, means that the substance from which the composition is free of cannot be detected by methods known to those skilled in the art.

Nateglinide has the chemical structure

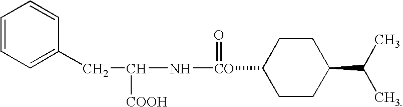

Its preparation is disclosed, for example, in U.S. Pat. No. 4,816,484, which is incorporated herein by reference in its entirety, and specifically for the purpose of showing how nateglinide is prepared and characterized. An article by Shinkai, et al., Journal of Medicinal Chemistry, 32:1436 1989, incorporated herein by reference specifically for the purpose of showing how nateglinide is prepared, also discloses the preparation of nateglinide and its related compounds generically, and their use as pharmaceuticals.

Different solid forms of the same drug may exhibit different properties, including characteristics that have functional implications with respect to their use as active ingredients of pharmaceutical products. For example, polymorphs of the same drug may have substantial differences in such pharmaceutically important properties as dissolution rates and bioavailability. Likewise, different polymorphs may have different processing properties, such as hydroscopicity, flowability, and the like, which could affect their suitability as active pharmaceuticals for commercial production. Two polymorphic forms of nateglinide, designated B and H, are disclosed in U.S. Pat. Nos. 5,488,150 and 5,463,116, which are incorporated herein by reference in their entirety, and specifically for the purposes of showing how the Forms B and H are prepared and characterized.

A new crystalline form of nateglinide has now been discovered. While the invention is not limited to any specific theory or preparation methodology, the inventors found that crystallization of nateglinide from aromatic hydrocarbon solvent produces a polymorph that is different from known polymorphs B and H. The new polymorph was designated as the crystalline Form X of nateglinide. The preparation of the crystalline Form X is described in greater details below. The new crystalline Form X may be identified and differentiated by X-ray diffraction and/or infrared spectroscopy.

The crystalline Form X of nateglinide may be characterized by X-ray powder diffraction. The X-ray diffraction patterns are unique for the particular crystalline form. Each crystalline form exhibits a diffraction pattern with a unique set of diffraction peaks that can be expressed in 2 theta angles, d-spacing values and relative peak intensities. 2 Theta diffraction angles and corresponding d-spacing values account for positions of various peaks in the X-ray powder diffraction pattern. D-spacing values are calculated with observed 2 theta angles and copper K(α1) wavelength using the Bragg equation well known to those of skill in the art.

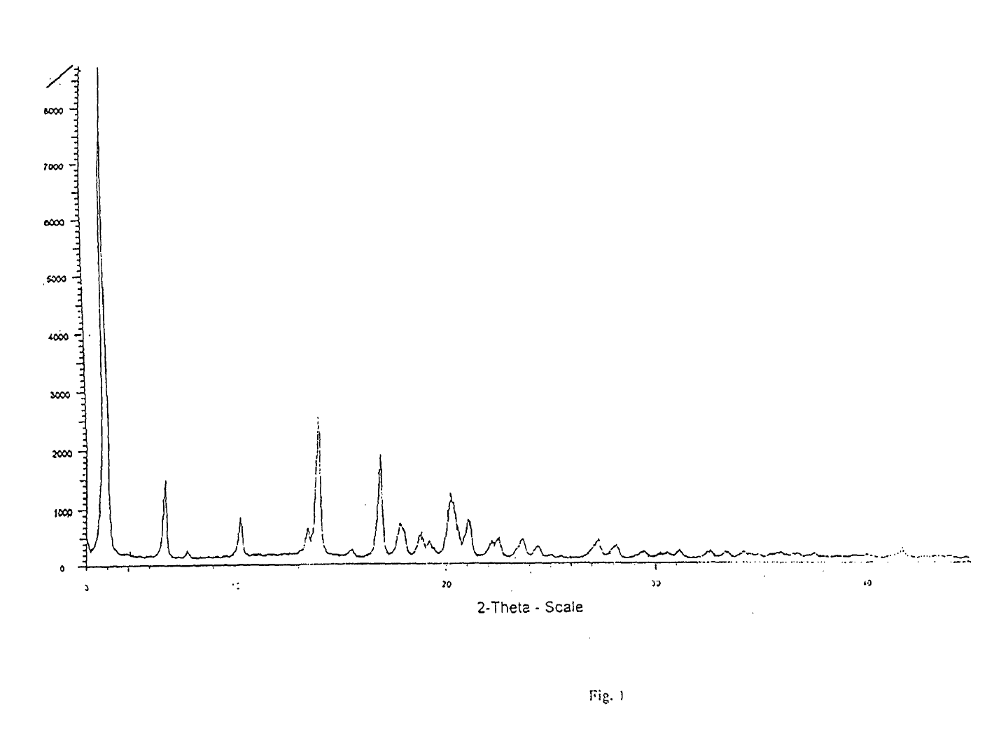

FIG. 1 shows an example of X-ray powder diffractogram of the crystalline Form X of netaglinide obtained on a Bruker Axs, D8 Advance Powder X-ray Diffractometer with Cu K alpha-1 Radiation source. The pattern of X-ray diffraction peaks for crystalline Form X of nateglinide is shown in Table 1:

TABLE 1

| 2-theta values | Relative Intensity (%) |
|---|---|
| 3.952 | 100 |
| 14.039 | 21.2 |
| 16.98 | 12.4 |
| 20.325 | 11.7 |
| 21.120 | 6.7 |
| 17.942 | 6.6 |
| 6.776 | 6.0 |
| 13.515 | 4.0 |
| 18.853 | 3.9 |
| 10.324 | 3.8 |
| 22.518 | 3.1 |
| 7.79 | 2.6 |
| 23.761 | 2.5 |
| 27.361 | 2.1 |
| 19.167 | 1.9 |
| 4.895 | 1.8 |
| 28.169 | 1.8 |
| 24.463 | 1.6 |
| 5.181 | 1.1 |
| 31.252 | 1.1 |
| 32.607 | 0.9 |
| 41.651 | 0.9 |
| 30.878 | 0.5 |

It should be kept in mind that slight variations in observed 2 theta angles or d-spacing values are expected based on the specific diffractometer employed, the analyst, and the sample preparation technique. More variation is expected for the relative peak intensities. Identification of the exact crystal form of a compound should be based primarily on observed 2 theta angles with lesser importance attributed to relative peak intensities. Table 2 shows another measurement of the X-ray powder diffraction pattern for crystalline Form X of nateglinide:

TABLE 2

| 2-theta values | Relative Intensity (%) |
|---|---|
| 3.860 | 100 |
| 13.945 | 27.7 |
| 16.894 | 19.7 |
| 6.674 | 15.2 |
| 20.24 | 11.6 |
| 10.223 | 7.7 |
| 21.072 | 7.3 |
| 17.836 | 6.7 |
| 13.398 | 5.2 |
| 18.767 | 4.0 |
| 22.473 | 3.7 |
| 27.284 | 3.5 |
| 23.653 | 3.4 |
| 22.166 | 2.7 |
| 19.170 | 2.6 |
| 28.137 | 2.4 |
| 24.387 | 1.9 |
| 7.7360 | 1.3 |
| 15.495 | 1.3 |
| 31.161 | 1.2 |
| 29.171 | 1.1 |
| 32.637 | 1.1 |
| 41.7440 | 1.0 |
| 34.229 | 1.0 |
| 33.403 | 0.9 |
| 35.997 | 0.8 |
| 37.560 | 0.8 |
| 30.574 | 0.6 |
| 36.788 | 0.6 |
| 30.207 | 0.5 |

As clear from a comparison between Tables 1 and 2, some margin of error is present in each of the 2 theta angle assignments reported herein. On this basis, the assigned margin of error in the 2 theta angles for Form X of nateglinide is approximately ±0.09 for each of the peak assignments. In view of the assigned margin of error, in a preferred variant, the crystalline Form X of nateglinide may be characterized by an X-ray powder diffraction patterns that includes five or more peaks selected from the group consisting of peaks with 2 theta angles of 3.95±0.09, 4.89±0.09, 5.18±0.09, 6.78±0.09, 7.79±0.09, 10.32±0.09, 13.51±0.09, 14.04±0.09, 16.98±0.09, 17.94±0.09, 18.85±0.09, 19.17±0.09, 20.32±0.09, 21.12±0.09, 22.52±0.09, 23.76±0.09, 24.46±0.09, 27.36±0.09, 28.17±0.09, 30.88±0.09, 31.25±0.09, 32.61±0.09, and 41.65±0.09. In particular the X-ray diffraction pattern could be expected to include peaks at 3.95±0.09, 14.00±0.09, and 16.98±0.09 degrees.

Since some margin of error is possible in the assignment of 2 theta angles and d-spacings, the preferred method of comparing X-ray powder diffraction patterns in order to identify a particular crystalline form is to overlay the X-ray powder diffraction pattern of the unknown form over the X-ray powder diffraction pattern of a known form. For example, one skilled in the art can overlay an X-ray powder diffraction pattern of an unidentified crystalline form of nateglinide obtained using the methods described herein, over FIG. I and readily determine whether the X-ray diffraction pattern of the unidentified form is substantially the same as the X-ray powder diffraction pattern of Form X. If the X-ray powder diffraction pattern is substantially the same as FIG. 1, the previously unknown crystalline form of netaglinide can be readily and accurately identified as Form X.

Although 2 theta angles or d-spacing values are the primary methods of identifying the crystalline form, it may be desirable to also compare relative peak intensities. As noted above, relative peak intensities may vary depending upon the specific diffractometer employed and the analyst's sample preparation technique. The peak intensities are reported as intensities relative to the peak intensity of the strongest peak.

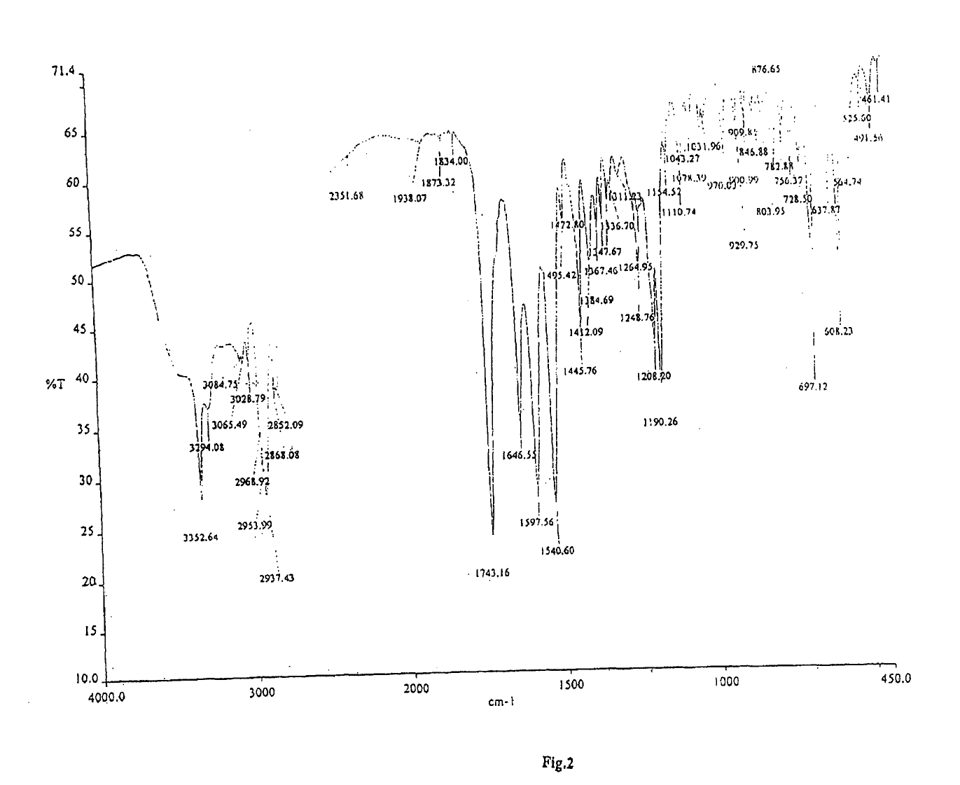

The crystalline form of nateglinide may be also characterized by infrared spectroscopy. The infrared spectrum of crystalline Form X of nateglinide obtained by the inventors is shown in FIG. 2. It was measured on Perkin-Elmer FT-IR instrument by KBr transmission method. The significant bands may be identified at about 3353 cm⁻¹, about 2937 cm⁻¹, about 2868 cm⁻¹, about 1743 cm⁻¹, about 1646 cm⁻¹, about 1597 cm⁻¹, about 1541 cm⁻¹, about 1445 cm⁻¹, about 1208 cm⁻¹, about 1190 cm⁻¹, about 1110 cm⁻¹, about 697 cm⁻¹, and about 607 cm⁻¹.

The invention also provides a composition containing solid nateglinide, of which at least 80%, by total weight of the solid nateglinide in the composition, is its crystalline Form X. The preferred form of this composition is solid nateglinide powder suitable for use as active ingredient in formulating pharmaceutical products. The remainder of the solid nateglinide in the composition, i.e., 20% or less of the total weight of nateglinide may be, for example, crystalline Forms B and/or H of nateglinide. In one specific embodiment, the composition contains at least 90% by weight of the crystalline Form X of nateglinide with respect to total weight of the solid nateglinide in the composition. In another specific embodiment, the composition contains at least 95% by weight of the crystalline Form X of nateglinide with respect to total weight of the solid nateglinide in the composition. In another embodiment, the composition contains at least 99% by weight of the crystalline Form X of nateglinide with respect to the total weight of the solid nateglinide in the composition. In yet another embodiment, the composition is substantially free of any forms of nateglinide other than its crystalline Form X. In yet another embodiment, in addition to crystalline Form X, the composition includes at least a small amount of crystalline Forms B or H of nateglinide, or both. In a non-limiting example, the composition includes 95% of crystalline Form X of nateglinide and at least 1% of other crystalline forms of nateglinide. In another non-limiting example, the composition includes at least 80% of crystalline Form X of nateglinide and at least 5 % of crystalline Forms B and/or H of nateglinide. All compositions, in 0.1% increments, which include at least 80% of crystalline Form X nateglinide and at least 1 % of other crystalline forms of nateglinide, are contemplated. All percentages are based upon the total amount of the solid nateglinide in the composition.

X-ray diffraction provides a convenient and practical means for quantitative determination of the relative amounts of crystalline and/or amorphous forms in a solid mixture. X-ray diffraction is adaptable to quantitative applications because the intensities of the diffraction peaks of a given compound in a mixture are proportional to the fraction of the corresponding powder in the mixture. The percent composition of crystalline nateglinide can be determined in an unknown composition. Preferably, the measurements are made on solid powder nateglinide. The X-ray powder diffraction patterns of an unknown composition can be compared to known quantitative standards containing pure crystalline forms of nateglinide (e.g., Forms B, H. or X) to identify the percent ratio of the crystalline form of nateglinide. This is done by comparing the relative intensities of the peaks from the diffraction pattern of the unknown solid powder composition with a calibration curve derived from the X-ray diffraction patterns of pure known samples. The curve can be calibrated based on the X-ray powder diffraction pattern for the strongest peak from a pure sample of crystalline nateglinide. The calibration curve may be created in a manner known to those of skill in the art. For example, five or more artificial mixtures of crystalline forms of nateglinide, at different amounts, may be prepared. In a non-limiting example, such mixtures may contain, 2%, 5%, 7%, 8%, and 10% of nateglinide for each crystalline form. Then, X-ray diffraction patterns are obtained for each artificial mixture using standard X-ray diffraction techniques. Slight variations in peak positions, if any, may be accounted for by adjusting the location of the peak to be measured. The intensities of the selected characteristic peak(s) for each of the artificial mixtures are then plotted against the known weight percentages of the crystalline form. The resulting plot is a calibration curve that allows determination of the amount of crystalline nateglinide in an unknown sample. For the unknown mixture of crystalline and amorphous nateglinide, the intensities of the selected characteristic peak(s) in the mixture, relative to an intensity of this peak in a calibration mixture, may be used to determine the percentage of the given crystalline form in the composition, with the remainder determined to be the amorphous material.

An improved process for the synthesis of N-(trans-4-isoproplycyclohexylcarbonyl)-D-phenylalanine also was found by the inventors. The object of the improved process was to provide a cost effective, substantially pure, easily scaleable, environmentally friendly process. The chemical route to prepare nateglinide is known and described in EP 0196222 and U.S. Pat. No. RE 34,878, which are hereby incorporated by reference in their entirety. The inventors found that the synthesis of nateglinide can be achieved by the condensation of D-phenyl alanine methyl ester with trans-4-isopropylcyclohexylcarboxylic acid chloride in the presence of a halo alkane solvent. Inexpensive solvents and reagents can be used in this process. For example methanol, chloroform and isopropanol are suitable solvents. Similarly, a suitable reagent is triethylamine thionyl chloride, which is also cost efficient. The solvents used in this process can be recovered and reused, making this process both economical and environmentally friendly.

The preparation described in the prior art is not economical for large-scale synthesis due to the expensive reagents and the effluent byproducts. The production of substantially pure nateglinide is problematic because of the formation of byproducts derived from the impurities present in the starting materials. The yields and purity of the ester intermediates are low and consequently, the nateglinide compound that is produced is low in purity due to contamination with the opposite L-enantiomer. Additional pharmaceutical steps are required to render the Nateglinide compound produced pharmaceutically acceptable.

In one variant of this aspect of the invention, the process for the preparation of N-(trans-4-isopropyl cyclohexyl-1-carbonyl)-D-phenyl alanine methyl ester which includes:

a) reacting 4-trans-isopropyl cyclohexyl carbonyl chloride with D-phenyl alanine methyl ester hydrochloride in halo alkane solvent such as chloroform or methylene chloride, preferably chloroform in the presence of C₁-C₄ alkyl tertiary amines, such as triethylamine;

b) cooling the reaction solution of step (a) to a temperature of 20-60° C., preferably 25-35° C.;

c) washing the reaction solution obtained in step (b) with 1N HCl, thereby removing D-phenyl alanine methyl ester;

d) separating the reaction solution of step (c) and distilling the excess chloroform solvent under reduced pressure;

e) dissolving the compound of step (d) in C₁-C₄ alcohol, preferably methanol, accompanied by cooling the resulting reaction mass to a temperature of 0-10° C.;

f) filtering off the solid and optionally washing the reaction mass with C₁-C₄ alcohol, preferably methanol;

g) dissolving the resulted reaction mass of step (e) in C₁-C₄ alcohol, preferably methanol;

h) distilling off the solvent from the reaction solution of step (g) accompanied by cooling the resulting reaction mass to precipitate the N-(trans 4-isopropyl cyclohexyl-1-carbonyl)-D-phenyl alanine methyl ester;

i) filtering the compound obtained in step (h) followed by drying the compound at temperature of 30-100° C. to afford N-(trans 4-isopropyl cyclohexyl-1-Carbonyl)-D-phenylalanine.

In another variant of this aspect of the invention, the process for the preparation of N-(trans-4-isopropyl cyclohexyl-1-carbonyl)-D-phenylalanine includes:

a) reacting 4-trans-isopropyl cyclohexyl carbonyl chloride with D-phenyl alanine methyl ester with 1N aqueous metal hydroxides such as sodium hydroxide in the presence of C₁-C₄ alcohol, such as isopropanol;

b) cooling the reaction solution of step (a) to a temperature of 20-40° C., preferably 25-35° C. accompanied by the treatment of the reaction solution with HCl to adjust the pH to 1.5 to 2.5 to result N-(trans-4-isopropyl cyclohexyl-1-carbonyl)-D-phenyl alanine;

c) filtering the crystalline salt at a temperature of 0-35° C., preferably 0-10° C. to afford N-(trans-4-isopropyl cyclohexyl-1-carbonyl)-D-phenyl alanine;

d) distilling off the solvent from the reaction solution of step (c) at 90° C. under vacuum to afford the N-(trans 4-isopropyl cyclohexyl-1-Carbonyl)-D-phenyl alanine which is substantially fee of the opposite L-enantiomer and cis-isomer.

A process for preparation of the crystalline Form X of nateglinide is also provided. As discussed above, the inventors had found that a new polymorph of nateglinide results from crystallizing nateglinide from an aromatic hydrocarbon solvent. Thus, a process for preparation of a crystalline Form X of nateglinide may include a) providing or forming a solution of nateglinide in an aromatic hydrocarbon solvent; b) cooling the solution until a precipitate is formed; and c) isolating the precipitate. Examples of aromatic hydrocarbons include benzene, naphthalene, anthracene, furan, thiophene, pyrroles, oxazoles, thiazoles, triazoles, imidazoles, pyridazine, pyridine, purines, pyrimidine, triazine, thiazine, indoles, quinolines, indenes, azulene, porphines, and any of the above rings which are fused with other rings or substituted. Preferred aromatic hydrocarbons are benzene and substituted benzenes, the substituted benzenes preferably substituted with an alkyl group. More preferred are unsubstituted benzene and methyl or dimethyl substituted benzenes (toluene and xylene). Xylene has three positional isomers, ortho, meta, and para xylene all of which are suitable. Xylene is available in the form of “mixed xylene”, which contains meta-xylene, para-xylene, ortho-xylene, and ethylbenzene. Most preferred are benzene, ethylbenzene, toluene, and orthoxylene.

The step of providing the solution of netaglinide may involve, for example, mixing netaglinide powder (of any type and in any form, crystalline or amorphous) with the aromatic hydrocarbon solvent, and heating the mixture until a solution is formed. Any ratio of the amount of the starting nateglinide to the solvent may be employed; however, preferred ratio is from about 5 milliliters of solvent per 1 gram of solid netaglinide to about 30 milliliters of solvent per gram; more preferably, from about 10 to about 20 milliliters of solvent per 1 gram of solid netaglinide. In one embodiment the starting netaglinide is crystalline Form H, crystalline Form B or a mixture thereof. Depending on the solvent and the ratio, the mixture may be heated to a temperature of about 40° C. to about 130° C., typically, to from about 60° C. to about 70° C. After the solution is formed, it may be filtered to remove extraneous matter. Subsequently, the solution is cooled to precipitate the desired product; typically, to a temperature of about 20° C. to about 60° C., preferably, to an ambient temperature (about 25° C.-35° C.). The precipitate may be washed with an aromatic hydrocarbon solvent, preferably, the same solvent that was used for re-crystallization. The isolated precipitate is then dried in conventional manner.

Also provided are pharmaceutical compositions containing a crystalline Form X of nateglinide and a pharmaceutically-acceptable carrier. In addition to the active compound, the pharmaceutical composition includes one or more pharmaceutically acceptable carriers, also known as excipients, which ordinarily lack pharmaceutical activity, but have various useful properties which may, for example, enhance the stability, sterility, bioavailability, and ease of formulation of a pharmaceutical composition. These carriers are pharmaceutically acceptable, meaning that they are not harmful to humans or animals when taken appropriately and are compatible with the other ingredients in a given formulation. The carriers may be solid, semi-solid, or liquid, and may be formulated with the compound in bulk, but ultimately in the form of a unit-dose formulation (i.e., a physically discrete unit containing a specific amount of active ingredient) such as a tablet or capsule. The pharmaceutical compositions may include, in addition to a compound of this invention, one or more active pharmaceutical compounds.

Generally, the pharmaceutical compositions are prepared by uniformly admixing the active ingredient with liquid or solid carriers and then shaping the product into the desired form. The pharmaceutical compositions may be in the form of suspensions, solutions, elixirs, aerosols, or solid dosage forms. Because of their ease of administration, tablets and capsules represent the most advantageous oral dosage unit form, in which case solid pharmaceutical carriers are employed.

The more preferred oral solid preparation is a tablet. A tablet may be prepared by direct compression, wet granulation, or molding, of the active ingredient(s) with a carrier and other excipients in a manner known to those skilled in the art. Compressed tablets may be prepared by compressing in a suitable machine the active ingredient in a free-flowing form such as powder or granules, optionally mixed with a binder, lubricant, inert diluent, surface active agent or dispersing agent. Molded tablets may be made on a suitable machine, a mixture of the powdered compound moistened with an inert liquid diluent are suitable in the case of oral solid dosage forms (e.g., powders, capsules, and tablets). If desired, tablets may be coated by standard techniques. The compounds of this invention may be formulated into typical disintegrating tablet, or into a controlled or extended release dosage forms. Examples of suitable controlled release formulation vehicles are disclosed in U.S. Pat. Nos. 3,845,770; 3,916,899; 3,536,809; 3,598,123; and 4,008,719, incorporated herein by reference in their entireties.

The pharmaceutical compositions are contemplated in various formulations suitable for various modes of administration, including but not limited to inhalation, oral, rectal, parenteral (including subcutaneous, intradermal, intramuscular, intravenous), implantable, intravaginal and transdermal administration. The most suitable route of administration in any given case depends on the duration of the subject's condition, the length of treatment desired, the nature and severity of the condition being treated, and the particular formulation that is being used. The formulations may be in bulk or in unit dosage form, and may be prepared by methods well known in the art for a given formulation.

The amount of active ingredient included in a unit dosage form depends on the type of formulation in which the active ingredient is presented. A pharmaceutical composition will generally contain about 0.1% by weight to about 99% by weight of active ingredient, preferably about 1% by weight to 50% by weight for oral administration and about 0.2% by weight to about 20% by weight for parenteral administration.

Formulations suitable for oral administration include capsules (hard and soft), cachets, lozenges, syrups, suppositories, and tablets, each containing a predetermined amount of the active compound; as a powder or granules; as a solution or a suspension in an aqueous or non-aqueous liquid; or as an oil-in-water or water-in-oil emulsion. Such formulations may be prepared by any suitable method of pharmacy that includes the step of bringing into association the active compound and a suitable carrier or carriers. Preferred oral or internal dosage forms may include, for example, between 1 mg and 1000 mg of nateglinide. The amount of active ingredient per unit dosage of solid formulations is preferably from about 40 mg to about 70 mg, preferably about 60 mg, about 140 mg to about 200 mg, preferably about 160 mg and about 180 mg. For liquid oral formulations, a preferable amount is from about 2% by weight to about 20% by weight. Suitable carriers include but are not limited to fillers, binders, lubricants, inert diluents, surface active/dispersing agents, flavorants, antioxidants, bulking and granulating agents, adsorbants, preservatives, emulsifiers, suspending and wetting agents, glidants, disintegrants, buffers and pH-adjusting agents, and colorants. Examples of carriers include celluloses, modified celluloses, cyclodextrins, starches, oils, polyols, sugar alcohols and sugars, and others. For liquid formulations sugar, sugar alcohols, ethanol, water, glycerol, and poyalkylene glycols are particularly suitable, and may also be used in solid formulations. Cyclodextrins may be particularly useful for increasing bioavailability. Formulations for oral administration may optionally include enteric coatings known in the art to prevent degradation of the formulation in the stomach and provide release of the drug in the small intestine. Example of suitable nateglinide dosage form is disclosed in U.S. Pat. No. 6,559,188, which is incorporated herein by reference in its entirety and for purposes of showing doses of nateglinide and formulation methodologies.

Formulations suitable for buccal or sub-lingual administration include lozenges comprising the active compound in a flavored base, usually sucrose and acacia or tragacanth, although other agents are also suitable, and pastilles comprising the compound in an inert base such as gelatin and glycerin or sucrose and acacia.

Formulations suitable for parenteral administration comprise sterile aqueous and non-aqueous injection solutions of the active compound, preferably isotonic with the blood of the intended recipient. The amount of active ingredient is preferably now about 0.1% by to about 80% by weight.

These preparations may contain, among other ingredients, anti-oxidants, buffers, bacteriostats, and solutes that render the formulation isotonic with the blood of the intended recipient. Aqueous and non-aqueous sterile suspensions may include, among others, suspending and thickening agents. The formulations may be presented in unit-dose or multi-dose containers, e.g., sealed capsules and vials, and may be stored in a freeze-dried or lyophilized condition requiring only the addition of the sterile liquid carrier, for example, saline or water-for-injection immediately prior to use. Extemporaneous injection solutions and suspensions may be prepared from sterile powders, granules and tablets of the kind previously described.

Formulations suitable for rectal administration are preferably presented as unit dose suppositories. These may be prepared by admixing the active compound with one or more conventional solid carriers, e.g., cocoa butter, and then shaping the resulting mixture.

Formulations suitable for transdermal delivery include ointments, creams, lotions, and oils and contain well-known pharmaceutically and cosmetically suitable ingredients. Bases for such formulations include for example alcohols, lanolin, petrolatum, paraffin, polyethylene glycol, emulsifiers, penetration enhancing agents, and oleaginous vehicles such as oils. Skin patches may also be used, typically consisting of a fabric or paper base impregnated with a suitable dose in a transdermal formulation. Formulations suitable for transdermal administration may also be delivered by iontophoresis, and typically take the form of an optionally buffered aqueous solution of the active compound.

The compounds of this invention may be combined with or linked to other compounds to obtain desired properties, for example the compounds of this invention may be linked to a stabilizing polymer such as a polyalkylene glycol (such as polyethylene glycol), or linked to a targeting compound such as an antibody. The resulting linked compounds are also part of this invention.

In another aspect, the invention also provides methods of treatment using the compounds and the pharmaceutical compositions of this invention. The compounds and compositions of this invention may be administered to a subject in an amount effective to stimulate insulin release by said subject. Further, the compounds and compositions of this invention may be administered to a subject for treating a disorder related to insulin release by administering to a subject an amount effective to stimulate insulin release by said subject. Methods for treating diabetes in a subject by administering a compound or composition of this invention to a subject in an amount effective to eliminate or alleviate symptoms of diabetes, or to prevent excessive blood sugar levels or reduce blood sugar levels, are also part of this invention. Methods for regulating blood sugar levels in a subject by administering an amount of a compound or composition of this invention effective to regulate blood sugar levels in the subject are also part of this invention.

In general, the treatment may be determined to alleviate, to eliminate, or to prevent a given condition based on factors determinable by a skilled physician as discussed below in the context of determining an effective amount for dosage.

By subject is meant a human or an animal, preferably human. Animals contemplated by this invention include any animal safely treatable by compounds of this invention, preferably mammals such as bovines, ovines, caprines, equines, felines, canines, rodents, leporids, and other mammalian farm and zoo animals or domestic pets.

The effective amount (i.e., dosage) of active compound for treatment will vary depending on the route of administration, the condition being treated, its severity, and duration, and the state and age of the subject. A skilled physician will monitor the progress of the subject and will adjust the dosage accordingly, depending on whether the goal is to eliminate, alleviate, or prevent a given condition. Generally, the dosage should be considered in proportion to the subject's weight. Depending on the solubility of the particular formulation of active compound administered, the daily dose may be divided among one or several unit dose administrations. For example therapeutic administration about fifteen to thirty minutes before main meals is preferable (i.e. three times daily), although administration of the active compounds may be carried out prophylactically, and may be maintained for prolonged periods of time. One skilled in the art will take such factors into account when determining dosage. In general dosages will be in the range of about 60 mg three times daily to about 180 mg three times daily.

The examples that follow are not intended to limit the scope of the invention as defined hereinabove or as claimed below.

### EXAMPLE 1

H-type crystals of N-(trans-4-isopropylcyclohexane carbonyl)-D-phenylalanine (10.0 g) were dissolved in xylene (150 ml) at a temperature of 50-70° C. and stirred for 1-2 hours. The resulting clear solution was filtered to remove extraneous matter. The clear filtrate was cooled to 25-35° C. under stirring to precipitate the compound. The resulting precipitated compound was filtered, washed with xylene (50.0 ml) and dried at a temperature of 60-70° C. under reduced pressure to a constant weight to provide crystalline form X N-(trans-4-isopropylcyclohexane carbonyl)-D-phenylalanine. (yield: 9.2 grams, 92.0%).

### EXAMPLE 2

H-type crystals of N-(trans-4-isopropylcyclohexane carbonyl)-D-phenylalanine (10.0 g) were dissolved in xylene (150 ml) at a temperature of 50-70° C. for 1-2 hours. The resulting clear solution was filtered to remove extraneous matter. The clear filtrate was cooled to 25-35° C. under stirring to precipitate the compound. The resulting precipitated compound was filtered, washed with xylene (50.0 ml) and dried at a temperature of 60-70° C. under reduced pressure to a constant weight to provide crystalline form X N-(trans-4-isopropylcyclohexane carbonyl)-D-phenylalanine. (Yield 8.3 grams, 83.0%).

### EXAMPLE 3

B-type crystals of N-(trans-4-isopropylcyclohexane carbonyl)-D-phenylalanine (10.0 g) were dissolved in ortho-xylene (150 ml) at a temperature of 50-70° C. and stirred for 1-2 hours. The resulting clear solution was filtered to remove extraneous matter. The clear filtrate was cooled to 25-35° C. under stirring to precipitate the compound. The resulting precipitated compound was filtered, washed with orthoxylene (50.0 ml) and dried at a temperature of 60-70° C. under reduced pressure to a constant weight to provide crystalline form X N-(trans-4-isopropylcyclohexane carbonyl)-D-phenylalanine. (Yield: 9.1 grams, 91 %).

### EXAMPLE 4

B-type crystals of N-(trans-4-isopropylcyclohexane carbonyl)-D-phenylalanine (8.0 g) were dissolved in xylene (120 ml) at a temperature of 50-70° C. for 1-2 hours. The resulting clear solution was filtered to remove extraneous matter. The clear filtrate was cooled to 25-35° C. under stirring to precipitate the compound. The resulting precipitated compound was filtered, washed with xylene (40.0 ml) and dried at a temperature of 60-70° C. under reduced pressure to a constant weight to provide crystalline form X N-(trans-4-isopropylcyclohexane carbonyl)-D-phenylalanine. (yield: 7.1 grams, 88.7%).

### EXAMPLE 5

A mixture of H-type and B-type crystals of N-(trans-4isopropylcyclohexane carbonyl)-D-phenylalanine (10.0 g) were dissolved in xylene (150 ml) at a temperature of 50-70° C. and stirred for 1-2 hours. The resulting clear solution was filtered to remove extraneous matter. The filtrate was cooled to 25-35° C. under stirring to precipitate the compound. The resulting precipitated compound was filtered, washed with xylene (50.0 ml) and dried at a temperature of 60-70° C. under reduced pressure to a constant weight to provide crystalline form X N-(trans-4-isopropylcyclohexane carbonyl)-D-phenylalanine. (Yield 9.2 grams, 92%).

### EXAMPLE 6

### N-(trans-4 isopropyl cyclohexyl carbonyl)-D-phenylalanine methyl ester

50 grams (0.294 moles) of trans-4-isopropylcyclohexylcarboxylic acid and 71.0 grams (0.50 moles) of Thionyl chloride was dissolved in 200 ml of chloroform at room temperature. The reaction solution was stirred for 6 hours at an ambient temperature and the excess thionyl chloride was distilled off under reduced pressure to obtain an oily residue. The resultant residue was dissolved in 100 ml chloroform and added to a solution containing 64.0 grams (0.29 moles) of D-Phenyl alanine methyl ester hydrochloride and 75.0 grams (0.74 moles) of Triethylamine in 500 ml chloroform. The reaction solution was stirred and maintained at an ambient temperature for 10 hours. The reaction solution was then washed under vacuum with 270 ml of a 1N HCL solution to evaporate the chloroform under vacuum to get residue. 350 ml of methanol was added to the residue and the reaction product was cooled to 0-10° C., and subsequently filtered and washed with 50 ml of methanol to afford 70.0 grams (71 %) of N-(trans-4 isopropyl-cyclohexyl carbonyl)-D-Phenyl alanine methyl ester, substantially free from cis content.

| M.R. | 123-129° C. |
|---|---|
| [a]25D | 7.7 |
| cis Isomer content | <0.05% |

### EXAMPLE 7

### N-(trans-4-isopropyl cyclohexyl carbonyl)-D-phenylalanine

60 grams (0.18 moles) of N-(trans-4-isopropyl-cyclohexyl carbonyl). D-phenylalanine methyl ester was dissolved in 300 ml of IN NaOH solution and stirred for 15-20 minutes at room temperature. 600 ml of isopropanol was added to the reaction solution and the reaction solution was stirred and maintained at an ambient temperature for 6 hours. 600 ml of water was added to the reaction solution and the pH of the solution was adjusted to 2.0 with HCL. The reaction mass was cooled, filtered and subsequently washed at 90° C. to obtain 53.7 grams (93.4 %) of N-(trans-4-isopropyl cyclohexyl carbonyl)-D-Phenylalanine, substantially free from cis and L-enantiomer impurities.

Cis isomer: <0.05%

L-enantiomer: <0.05%

Unless stated to the contrary, words and phrases such as “including,” “containing,” “comprising,” “having”, “for example”, “i.e.”, “in particular” and the like, means “including without limitation” and shall not be construed to limit any general statement that it follows to the specific or similar items or matters immediately following it. Except where the context indicates to the contrary, all exemplary values are intended to be used for purposes of illustration. Most of the foregoing alternative embodiments are not mutually exclusive, but may be implemented in various combinations. As these and other variations and combinations of the features discussed above can be utilized without departing from the invention as defined by the claims, the foregoing description of the embodiments should be taken by way of illustration rather than by way of limitation of the invention as defined by the appended claims.

1. A compound which is a crystalline Form X of nateglinide.

2. The compound of claim 1, having an X-ray diffraction pattern, expressed in terms of 2 theta angles, that includes five or more peaks selected from the group consisting of 3.95±0.09, 4.89±0.09, 5.18±0.09, 6.78±0.09, 7.79±0.09, 10.32±0.09, 13.51±0.09, 14.00±0.09, 16.98±0.09, 17.94±0.09, 18.85±0.09, 19.17±0.09, 20.32±0.09, 21.12±0.09, 22.52±0.09, 23.76±0.09, 24.46±0.09, 27.36±0.09, 28.17±0.09, 30.88±0.09, 31.25±0.09, 32.61±0.09, and 41.65±0.09 degrees.

3. The compound of claim 2, wherein said X-ray diffraction pattern includes at least the peaks at 3.95±0.09, 14.00±0.09, and 16.98±0.09 degrees.

4. The compound of claim 2, wherein said X ray diffraction pattern includes peaks at 3.952, 14.039, 16.98, 20.325, 21.120, 17.942, 6.776, 13.515, and 18.853 degrees.

5. The compound of claim 1, having an infrared absorption spectrum with absorption bands at about 3353 cm⁻¹, about 2937 cm⁻¹, about 2868 cm⁻¹, about 1743 cm⁻¹, about 1646 cm⁻¹, about 1597 cm⁻¹, about 1541 cm⁻¹, about 1445 cm⁻¹, about 1208 cm⁻¹, about 1190 cm⁻¹, about 1110 cm⁻¹, about 697 cm⁻¹, and about 607 cm⁻¹.

6. The compound of claim 1, having substantially the same X-ray diffraction pattern as that shown in FIG. 1.

7. The compound of claim 6, having substantially the same infrared spectrum as that shown in FIG. 2.

8. A composition comprising nateglinide as a solid, wherein at least 80% by weight of said solid nateglinide is its crystalline Form X having an X-ray diffraction pattern, expressed in terms of 2 theta angles, that includes five or more peaks selected from the group consisting of 3.95±0.09, 4.89±0.09, 5.18±0.09, 6.78±0.09, 7.79±0.09, 10.32±0.09, 13.51±0.09, 14.04±0.09, 16.98±0.09, 17.94±0.09, 18.85±0.09, 19.17±0.09, 20.32±0.09, 21.12±0.09, 22.52±0.09, 23.76±0.09, 24.46±0.09, 27.36±0.09, 28.17±0.09, 30.88±0.09, 31.25±0.09, 32.61±0.09, and 41.65±0.09.

9. The composition of claim 8, wherein said X-ray diffraction pattern includes at least the peaks at 3.95±0.09, 14.00±0.09, and 16.98±0.09 degrees.

10. The composition of claim 8, wherein at least 90% by weight of said solid nateglinide is its crystalline Form X.

11. The composition of claim 8, wherein at least 95% by weight of said solid nateglinide is its crystalline Form X.

12. The composition of claim 8, wherein at least 99% by weight of said solid nateglinide is its crystalline Form X.

13. The composition of claim 8, wherein said solid nateglinide is substantially free of its crystalline Forms H and B.

14. The composition of claim 8, wherein at least 1% of said solid nateglinide is not its crystalline Form X.

15. The composition of claim 8, wherein at least 5% of said solid nateglinide is not its crystalline Form X.

16. A pharmaceutical composition comprising the compound of claim 1 and a pharmaceutically acceptable carrier or diluent.

17. The pharmaceutical composition of claim 16, further comprising one or more pharmaceutically acceptable excipients.

18. The pharmaceutical composition of claim 17, which is a solid dosage form for oral administration.

19. The pharmaceutical composition of claim 18, wherein said solid dosage form is a tablet.

20. A process for preparation a crystalline form X of nateglinide, said process comprising:
a. providing a solution of nateglinide in an aromatic hydrocarbon solvent;
b. cooling the solution until a precipitate is formed; and
c. isolating the precipitate, which is the crystalline form X of nateglinide.

21. The process of claim 20, further comprising drying the isolated precipitate.

22. The process of claim 20, wherein said aromatic hydrocarbon solvent is selected from the group consisting of benzene, ethyl benzene, toluene, and xylene.

23. The process of claim 20, wherein said aromatic hydrocarbon solvent is xylene or ortho-xylene.

24. The process of claim 20, wherein the starting nateglinide is crystalline Form H, crystalline Form B, or a mixture thereof.

25. The process of claim 20, wherein said providing step includes heating a mixture of the starting nateglinide and the aromatic hydrocarbon solvent to a temperature of from about 40° C. to about 130° C. until the solution is formed.

26. The process of claim 25, wherein the mixture is heated to from about 60° C. to about 70° C.

27. The process of claim 20, further comprising filtering said provided solution of nateglinide prior to said cooling step.

28. The process of claim 20, wherein the solution of nateglinide is cooled to from about 25° C. to about 35° C.

29. A process for making crystalline form X of nateglinide, said process comprising:
a. forming a solution of nateglinide in xylene or ortho-xylene at from about 50° C. to about 70° C.;
b. cooling the solution from 25° C. to about 35° C. to form a precipitate; and
c. filtering said precipitate.

30. The process of claim 9, further comprising drying the precipitate.

31. A compound produced by the process of claim 20.

32. A compound produced by the process of claim 29.

33. A compound produced by the process of claim 30.
